SEQUENCE LISTING (19 sequences)

SEQ ID NO: 1 - protein, 22 aa, Geotrichum sp. M128
KEHYEFKNVAIGGGGYITGIVA

SEQ ID NO: 2 - protein, 12 aa, Geotrichum sp. M128
DLLYARTDIGGA

SEQ ID NO: 3 - protein, 12 aa, Geotrichum sp. M128
ASAPSAVFIWGT

SEQ ID NO: 4 - protein, 9 aa, Geotrichum sp. M128
VYGRVYLGT

SEQ ID NO: 5 - DNA, 23 bp, Artificial Sequence
  Primer
garcaytaygarttyaaraaygt

SEQ ID NO: 6 - DNA, 20 bp, Artificial Sequence
  Primer
gtnccccadatraanacngc

SEQ ID NO: 7 - DNA, 25 bp, Artificial Sequence
  Primer
cgtacagcaggtccttggtctttgg

SEQ ID NO: 8 - DNA, 22 bp, Artificial Sequence
  Primer
taatgtacccgccgccgccgat

SEQ ID NO: 9 - DNA, 24 bp, Artificial Sequence
  Primer
ggcaagttcttcgtctcgaccgac

SEQ ID NO: 10 - DNA, 24 bp, Artificial Sequence
  Primer
ccaagtcggacggcaagaaggcta

SEQ ID NO: 11 - DNA, 2646 bp, Geotrichum sp. M128
  CDS (120)..(2558)
aatcgccccggtgttggctcatcgggccgcggtgtcggtgttgatacgagtacataattgggactgtcggtgcgattggcccccaacttcaccacacaacacctacacactctttagatatggtggcagtcacctccctcgggaaggcgcttactgccctttcgattctggcgtcgctcgcggtcgccaaggagcactacgagttcaagaatgtcgcgatcggcggcggcgggtacattaccgggattgtcgcgcacccaaagaccaaggacctgctgtacgcgcgcacggacattggcggcgcgtaccgctgggacgcaggcacgtccaagtggatcccgctcaacgactttatcgaggcgcaggacatgaacattatgggcaccgagtcgatcgcgctggaccccaacaaccccgacaggctgtacctcgcgcaggggcgctatgtcggcgacgagtgggcggcgttctatgtgtccgaagaccgcggccagtcgtttacaatctacgagtcgccgttcccgatgggcgccaacgacatgggacgcaacaatggcgagcgcctcgctgtcaacccgttcaactcgaacgaggtctggatgggtacgcgtacagagggtatctggaagagttcggaccgcgccaagacctggacaaacgtcacgtccatcccggacgcgttcaccaacggtatcggatacacgtcggtcattttcgaccccgaacgtaatggcaccatctacgcgagcgcgactgccccgcagggcatgtacgtcacgcacgacggcggtgtctcgtgggagccagtggcgggccagccgtccagctggctcaacaggaccacgggcgcgttcccggacaagaagcccgcgtcgatcgcgccgcagcccatgaaagtcgctctcacccccaacttcctctacgtgacttacgccgactaccctggtccatggggcgtcacgttcggcgaagtctggcgccagaaccgcacctcgggcgcctgggacgacattactccccgcgtcggcaactcgtcgcctgccccgtacaacaaccagacgttccctgcgggcggattttgcggtctcagcgtcgacgcgaccaaccccaaccgtctcgtcgtcatcaccctcgaccgcgaccccggacccgccctcgacagcatctacctctcaaccgatgccggcgcgacctggaaggacgtcacccagctctcgtccccgtccaacctcgaaggtaactggggccacccgactaacgcggcgcggtacaaggacggcacgcctgttccgtggctcgacttcaacaacggtccccagtgggggggatacggtgcgccgcacggtacgcccggcctcaccaagtttggctggtggatgagcgctgtgcttatcgatccgttcaaccccgagcacctgatgtacggcacgggggcgaccatctgggcgaccgacacgctctcccgtgtcgagaaggactgggcgccgagctggtacctccagatcgacggtatcgaggagaatgcgatcctgtcgctccgctcgcccaagagcggcgcggcgctcctgtcgggcatcggtgacattagcggcatgaagcacgacgacctcaccaagccccagaagatgtttggtgcgccccagttctccaacctcgacagcatcgacgctgcgggcaacttccccaacgttgtcgtccgcgccggatcctcgggacacgagtacgacagcgcgtgcgcgcgcggtgcgtacgcgactgacggcggagacgcgtggaccatcttccctacctgccctcctggcatgaacgcgagccactaccagggcagcacgattgcagtcgacgcgagcggcagccagatcgtgtggtcgaccaagcttgacgagcaggcctcgggaccgtggtactcgcacgactatggcaagacgtggtctgttcccgctggcgacctgaaggcccagactgccaatgtgctctcggacaaggtccaggatggcacgttctacgctaccgatggcggcaagttcttcgtctcgaccgacggcgggaagtcgtatgccgccaagggcgccggacttgtcactggcacatcgctcatgcctgccgtgaacccctgggtggccggcgacgtctgggtgcctgttcccgagggcggtctcttccactcgaccgactttggcgcctcgttcacgagggtaggtaccgccaacgcgaccctcgtgagcgtcggcgcccccaagtccaagtcggacggcaagaaggctagcgcgccctccgcggtcttcatctggggcaccgacaagcctggaagcgacatcggcctgtaccgctccgacgacaacggcagcacctggacgcgcgtcaatgaccaggagcacaactactcgggccccaccatgatcgaggccgaccccaaggtctacgggcgcgtgtatctaggcacgaacggccgcggtatcgtgtacgccgaccttaccaacaagaagagcaacgaggagaagtcgaccgcaaagtgcgccaacggccagaagggcacgcactgctatgtgaaaaagtaaatgggaaggaaagggcgaaagggacataccgcctggttatttaaatgcatctacatgttacttaaaaaaaaaaaaaaaaaaaaaaaaa

SEQ ID NO: 12 - protein, 812 aa, Geotrichum sp. M128 [sequence omitted]

SEQ ID NO: 13 - DNA, 2367 bp, Geotrichum sp. M128
  mat_peptide (1)..(2367)
  CDS (1)..(2367)
aaggagcactacgagttcaagaatgtcgcgatcggcggcggcgggtacattaccgggattgtcgcgcacccaaagaccaaggacctgctgtacgcgcgcacggacattggcggcgcgtaccgctgggacgcaggcacgtccaagtggatcccgctcaacgactttatcgaggcgcaggacatgaacattatgggcaccgagtcgatcgcgctggaccccaacaaccccgacaggctgtacctcgcgcaggggcgctatgtcggcgacgagtgggcggcgttctatgtgtccgaagaccgcggccagtcgtttacaatctacgagtcgccgttcccgatgggcgccaacgacatgggacgcaacaatggcgagcgcctcgctgtcaacccgttcaactcgaacgaggtctggatgggtacgcgtacagagggtatctggaagagttcggaccgcgccaagacctggacaaacgtcacgtccatcccggacgcgttcaccaacggtatcggatacacgtcggtcattttcgaccccgaacgtaatggcaccatctacgcgagcgcgactgccccgcagggcatgtacgtcacgcacgacggcggtgtctcgtgggagccagtggcgggccagccgtccagctggctcaacaggaccacgggcgcgttcccggacaagaagcccgcgtcgatcgcgccgcagcccatgaaagtcgctctcacccccaacttcctctacgtgacttacgccgactaccctggtccatggggcgtcacgttcggcgaagtctggcgccagaaccgcacctcgggcgcctgggacgacattactccccgcgtcggcaactcgtcgcctgccccgtacaacaaccagacgttccctgcgggcggattttgcggtctcagcgtcgacgcgaccaaccccaaccgtctcgtcgtcatcaccctcgaccgcgaccccggacccgccctcgacagcatctacctctcaaccgatgccggcgcgacctggaaggacgtcacccagctctcgtccccgtccaacctcgaaggtaactggggccacccgactaacgcggcgcggtacaaggacggcacgcctgttccgtggctcgacttcaacaacggtccccagtgggggggatacggtgcgccgcacggtacgcccggcctcaccaagtttggctggtggatgagcgctgtgcttatcgatccgttcaaccccgagcacctgatgtacggcacgggggcgaccatctgggcgaccgacacgctctcccgtgtcgagaaggactgggcgccgagctggtacctccagatcgacggtatcgaggagaatgcgatcctgtcgctccgctcgcccaagagcggcgcggcgctcctgtcgggcatcggtgacattagcggcatgaagcacgacgacctcaccaagccccagaagatgtttggtgcgccccagttctccaacctcgacagcatcgacgctgcgggcaacttccccaacgttgtcgtccgcgccggatcctcgggacacgagtacgacagcgcgtgcgcgcgcggtgcgtacgcgactgacggcggagacgcgtggaccatcttccctacctgccctcctggcatgaacgcgagccactaccagggcagcacgattgcagtcgacgcgagcggcagccagatcgtgtggtcgaccaagcttgacgagcaggcctcgggaccgtggtactcgcacgactatggcaagacgtggtctgttcccgctggcgacctgaaggcccagactgccaatgtgctctcggacaaggtccaggatggcacgttctacgctaccgatggcggcaagttcttcgtctcgaccgacggcgggaagtcgtatgccgccaagggcgccggacttgtcactggcacatcgctcatgcctgccgtgaacccctgggtggccggcgacgtctgggtgcctgttcccgagggcggtctcttccactcgaccgactttggcgcctcgttcacgagggtaggtaccgccaacgcgaccctcgtgagcgtcggcgcccccaagtccaagtcggacggcaagaaggctagcgcgccctccgcggtcttcatctggggcaccgacaagcctggaagcgacatcggcctgtaccgctccgacgacaacggcagcacctggacgcgcgtcaatgaccaggagcacaactactcgggccccaccatgatcgaggccgaccccaaggtctacgggcgcgtgtatctaggcacgaacggccgcggtatcgtgtacgccgaccttaccaacaagaagagcaacgaggagaagtcgaccgcaaagtgcgccaacggccagaagggcacgcactgctatgtgaaaaag

SEQ ID NO: 14 - protein, 789 aa, Geotrichum sp. M128
KEHYEFKNVAIGGGGYITGIVAHPKTKDLLYARTDIGGAYRWDAGTSKWIPLNDFIEAQDMNIMGTESIALDPNNPDRLYLAQGRYVGDEWAAFYVSEDRGQSFTIYESPFPMGANDMGRNNGERLAVNPFNSNEVWMGTRTEGIWKSSDRAKTWTNVTSIPDAFTNGIGYTSVIFDPERNGTIYASATAPQGMYVTHDGGVSWEPVAGQPSSWLNRTTGAFPDKKPASIAPQPMKVALTPNFLYVTYADYPGPWGVTFGEVWRQNRTSGAWDDITPRVGNSSPAPYNNQTFPAGGFCGLSVDATNPNRLVVITLDRDPGPALDSIYLSTDAGATWKDVTQLSSPSNLEGNWGHPTNAARYKDGTPVPWLDFNNGPQWGGYGAPHGTPGLTKFGWWMSAVLIDPFNPEHLMYGTGATIWATDTLSRVEKDWAPSWYLQIDGIEENAILSLRSPKSGAALLSGIGDISGMKHDDLTKPQKMFGAPQFSNLDSIDAAGNFPNVVVRAGSSGHEYDSACARGAYATDGGDAWTIFPTCPPGMNASHYQGSTIAVDASGSQIVWSTKLDEQASGPWYSHDYGKTWSVPAGDLKAQTANVLSDKVQDGTFYATDGGKFFVSTDGGKSYAAKGAGLVTGTSLMPAVNPWVAGDVWVPVPEGGLFHSTDFGASFTRVGTANATLVSVGAPKSKSDGKKASAPSAVFIWGTDKPGSDIGLYRSDDNGSTWTRVNDQEHNYSGPTMIEADPKVYGRVYLGTNGRGIVYADLTNKKSNEEKSTAKCANGQKGTHCYVKK

SEQ ID NO: 15 - DNA, 40 bp, Artificial Sequence
  Primer
tcgctcgcgcatatgaaggagcactacgagttcaagaatg

SEQ ID NO: 16 - DNA, 38 bp, Artificial Sequence
  Primer
tggaagatctcctttttcacatagcagtgcgtgccctt

SEQ ID NO: 17 - DNA, 2481 bp, Artificial Sequence
  Xyloglucan Oligosaccharide-Degrading Enzyme with Histidine Tag
atgaaggagcactacgagttcaagaatgtcgcgatcggcggcggcgggtacattaccgggattgtcgcgcacccaaagaccaaggacctgctgtacgcgcgcacggacattggcggcgcgtaccgctgggacgcaggcacgtccaagtggatcccgctcaacgactttatcgaggcgcaggacatgaacattatgggcaccgagtcgatcgcgctggaccccaacaaccccgacaggctgtacctcgcgcaggggcgctatgtcggcgacgagtgggcggcgttctatgtgtccgaagaccgcggccagtcgtttacaatctacgagtcgccgttcccgatgggcgccaacgacatgggacgcaacaatggcgagcgcctcgctgtcaacccgttcaactcgaacgaggtctggatgggtacgcgtacagagggtatctggaagagttcggaccgcgccaagacctggacaaacgtcacgtccatcccggacgcgttcaccaacggtatcggatacacgtcggtcattttcgaccccgaacgtaatggcaccatctacgcgagcgcgactgccccgcagggcatgtacgtcacgcacgacggcggtgtctcgtgggagccagtggcgggccagccgtccagctggctcaacaggaccacgggcgcgttcccggacaagaagcccgcgtcgatcgcgccgcagcccatgaaagtcgctctcacccccaacttcctctacgtgacttacgccgactaccctggtccatggggcgtcacgttcggcaaagtctggcgccagaaccgcacctcgggcgcctgggacgacattactccccgcgtcggcaactcgtcgcctgccccgtacaacaaccagacgttccctgcgggcggattttgcggtctcagcgtcgacgcgaccaaccccaaccgtctcgtcgtcatcaccctcgaccgcgaccccggacccgccctcgacagcatctacctctcaaccgatgccggcgcgacctggaaggacgtcacccagctctcgtccccgtccaacctcgaaggtaactggggccacccgactaacgcggcgcggtacaaggacggcacgcctgttccgtggctcgacttcaacaacggtccccagtgggggggatacggtgcgccgcacggtacgcccggcctcaccaagtttggctggtggatgagcgctgtgcttatcgatccgttcaaccccgagcacctgatgtacggcacgggggcgaccatctgggcgaccgacacgctctcccgtgtcgagaaggactgggcgccgagctggtacctccagatcgacggtatcgaggagaatgcgatcctgtcgctccgctcgcccaagagcggcgcggcgctcctgtcgggcatcggtgacattagcggcatgaagcacgacgacctcaccaagccccagaagatgtttggtgcgccccagttctccaacctcgacagcatcgacgctgcgggcaacttccccaacgttgtcgtccgcgccggatcctcgggacacgagtacgacagcgcgtgcgcgcgcggtgcgtacgcgactgacggcggagacgcgtggaccatcttccctacctgccctcctggcatgaacgcgagccactaccagggcagcacgattgcagtcgacgcgagcggcagccagatcgtgtggtcgaccaagcttgacgagcaggcctcgggaccgtggtactcgcacgactatggcaagacgtggtctgttcccgctggcgacctgaaggcccagactgccaatgtgctctcggacaaggtccaggatggcacgttctacgctaccgatggcggcaagttcttcgtctcgaccgacggcgggaagtcgtatgccgccaagggcgccggacttgtcactggcacatcgctcatgcctgccgtgaacccctgggtggccggcgacgtctgggtgcctgttcccgagggcggtctcttccactcgaccgactttggcgcctcgttcacgagggtaggtaccgccaacgcgaccctcgtgagcgtcggcgcccccaagtccaagtcggacggcaagaaggctagcgcgccctccgcggtcttcatctggggcaccgacaagcctggaagcgacatcggcctgtaccgctccgacgacaacggcagcacctggacgcgcgtcaatgaccaggagcacaactactcgggccccaccatgatcgaggccgaccccaaggtctacgggcgcgtgtatctaggcacgaacggccgcggtatcgtgtacgccgaccttaccaacaagaagagcaacgaggagaagtcgaccgcaaagtgcgccaacggccagaagggcacgcactgctatgtgaaaaaggagatctgggtaccctggtgccacgcggttccatggctgatatcggatccgaattcgagctccgtcgacaagcttgcggccgcactcgagcaccaccaccaccaccactga

SEQ ID NO: 18 - protein, 826 aa, Artificial Sequence
  Xyloglucan Oligosaccharide-Degrading Enzyme with Histidine Tag
MKEHYEFKNVAIGGGGYITGIVAHPKTKDLLYARTDIGGAYRWDAGTSKWIPLNDFIEAQDMNIMGTESIALDPNNPDRLYLAQGRYVGDEWAAFYVSEDRGQSFTIYESPFPMGANDMGRNNGERLAVNPFNSNEVWMGTRTEGIWKSSDRAKTWTNVTSIPDAFTNGIGYTSVIFDPERNGTIYASATAPQGMYVTHDGGVSWEPVAGQPSSWLNRTTGAFPDKKPASIAPQPMKVALTPNFLYVTYADYPGPWGVTFGKVWRQNRTSGAWDDITPRVGNSSPAPYNNQTFPAGGFCGLSVDATNPNRLVVITLDRDPGPALDSIYLSTDAGATWKDVTQLSSPSNLEGNWGHPTNAARYKDGTPVPWLDFNNGPQWGGYGAPHGTPGLTKFGWWMSAVLIDPFNPEHLMYGTGATIWATDTLSRVEKDWAPSWYLQIDGIEENAILSLRSPKSGAALLSGIGDISGMKHDDLTKPQKMFGAPQFSNLDSIDAAGNFPNVVVRAGSSGHEYDSACARGAYATDGGDAWTIFPTCPPGMNASHYQGSTIAVDASGSQIVWSTKLDEQASGPWYSHDYGKTWSVPAGDLKAQTANVLSDKVQDGTFYATDGGKFFVSTDGGKSYAAKGAGLVTGTSLMPAVNPWVAGDVWVPVPEGGLFHSTDFGASFTRVGTANATLVSVGAPKSKSDGKKASAPSAVFIWGTDKPGSDIGLYRSDDNGSTWTRVNDQEHNYSGPTMIEADPKVYGRVYLGTNGRGIVYADLTNKKSNEEKSTAKCANGQKGTHCYVKKEIWVPWCHAVPWLISDPNSSSVDKLAAALEHHHHHH

SEQ ID NO: 19 - DNA, 18 bp, Artificial Sequence
  Histidine tag
caycaycaycaycaycay

(19) United States
(12) Patent Application Publication
(10) Pub. No.: US 2004/0038367 A1
(43) Pub. Date: Feb. 26, 2004

(54) Novel xyloglucan oligosaccharide-degrading enzyme, polynucleotide encoding the enzyme, and method of preparing the enzyme

(75) Inventors: Katsuro Yaoi, Ibaraki-ken (JP); Yasushi Mitsuishi, Ibaraki-ken (JP)

Correspondence Address: SUGHRUE MION, PLLC, 2100 PENNSYLVANIA AVENUE, N.W., WASHINGTON, DC, 20037, US

(73) Assignee: NATIONAL INSTITUTE OF ADVANCED INDUSTRIAL SCIENCE AND TECHNOLOGY

(21) Appl. No.: 10/395,241

(22) Filed: Mar. 25, 2003

(30) Foreign Application Priority Data
Mar. 25, 2002 (JP) 2002-83433

(51) Int. Cl. C12P 19/04; C07H 21/04; C08B 37/00; C12N 9/24; C12P 21/02; C12N 5/06

(52) U.S. Cl. 435/200; 435/69.1; 435/101; 435/320.1; 435/325; 536/23.2; 536/123

(57) ABSTRACT

The present invention provides a novel xyloglucan oligosaccharide-degrading enzyme having a different degradation mechanism from known enzymes. This enzyme specifically cleaves the second β-glucoside linkage, counted from the reducing end among the β-glucoside linkage constituting the principal chain of xyloglucan oligosaccharide. In the present invention, the enzyme is collected from microorganisms belonging to Geotrichum species, and the amino acid and base sequences of the enzyme and polynucleotide encoding the enzyme are determined. Thus, high-purity polypeptides having the xyloglucan oligosaccharide-degradation activity can be prepared at low cost through a genetic engineering process.

### FIELD OF THE INVENTION

The present invention relates to a novel xyloglucan oligosaccharide-degrading enzyme, a polypeptide comprising a specific amino acid sequence having a xyloglucan oligosaccharide-degradation activity, a polynucleotide encoding the polypeptide, a recombinant vector containing the polynucleotide, a transformant transformed by the recombinant vector, and a method of culturing the transformant to prepare the polypeptide.

### BACKGROUND OF THE INVENTION

Xyloglucan is a heteropolysaccharide comprising monosaccharides such as glucose, xylose, galactose, fucose and arabinose. The name “xyloglucan” is derived from its basic structure in which xylose branches formed by α-1,6 xyloside linkages are frequently linked to the principal chain having a number of glucoses linked thereto by β-1,4-glucoside linkages. Xyloglucan plays an important role as not only a component of the primary cell walls of a plant but also as a polysaccharide stored in seeds of tropical leguminous plants represented by Tamarindus indica, and its structure and function are regarded as noteworthy research subjects. In particular, it is believed that xyloglucan in the primary cell walls of a plant is closely related to cellulose, and its morphological status relative to cellulose is a critical factor in the elongation and/or morphological differentiation of plant cells. Among conventional techniques for use in analyzing the roles of xyloglucan, various types of glycosidases capable of degrading xyloglucan serve as an important and powerful analyzing tool.

The known glycosidases capable of degrading xyloglucan, which comprises monosaccharides such as glucose, xylose, galactose, fucose and arabinose and has a principal chain structure similar to cellulose, as described above, includes endo-1,4-β-D-glucanase, β-galactosidase, isoprimeverose-producing-oligoxyloglucan-degrading enzyme, α-xylosidase, β-glucosidase and α-fucosidase. These enzymes, originated from various sources, have been used to analyze the structure and function of xyloglucan.

However, as assumable from the complicated structure of xyloglucan, the known glycosidases do not cleave the linkages to completion, and a number of uncleaved linkages are left. Thus, the need for developing a novel glycosidase having a different degradation mechanism from the known enzymes still exists.

### SUMMARY OF THE INVENTION

In view of the fact that xyloglucan includes predominant glucoside linkages which cannot be cleaved directly by the known glycosidases and that enzymes generally have a high substrate specificity, the inventors carried out the screening of natural microorganisms capable of utilizing xyloglucan as a carbon source. Xyloglucan oligosaccharide substrates having various structures were used to screen the microorganisms for xyloglucan-degrading enzyme systems capable of degrading xyloglucan through unknown degradation mechanisms. As a result, the inventors found that one strain belonging to Geotrichum species (Geotrichum-species strain M128), a yeast fungi and a strain known to produced high-purity xyloglucan nona-oligosaccharide in large quantities, produces a novel xyloglucan oligosaccharide-degrading enzyme. Based on this knowledge, the inventors analyzed the amino acid sequence of the enzyme and the polynucleotide encoding the enzyme, thereby accomplishing the present invention.

Specifically, according to a first aspect of the present invention, there is provided an enzyme which specifically cleaves the second β-glucoside linkage, counted from the reducing end among the β-glucoside linkages constituting the principal chain of xyloglucan oligosaccharide.

According to a second aspect of the present invention, there is provided a polypeptide comprising an amino acid sequence shown in SEQ ID NO: 14, or an amino acid sequence having one or more amino acid deletions, additions, insertions or substitutions relative to the amino acid sequence shown in SEQ ID NO: 14. The polypeptide has a xyloglucan oligosaccharide-degradation activity.

The polypeptide set forth in the second aspect of the present invention may further include methionine at the N-terminus thereof.

The polypeptide set forth in the second aspect of the present invention may further include a signal sequence.

According to a third aspect of the present invention, there is provided a polypeptide comprising an amino acid sequence shown in SEQ ID NO: 12, or an amino acid sequence having one or more amino acid deletions, additions, insertions or substitutions relative to the amino acid sequence shown in SEQ ID NO: 12. This polypeptide serves as a precursor of a polypeptide having a xyloglucan oligosaccharide-degradation activity.

The amino acid sequence of the above polypeptide including methionine at its N-terminus in the second aspect of the present invention may be an amino acid sequence shown in SEQ ID NO: 18.

According to a fourth aspect of the present invention, there is provided a polynucleotide encoding the polypeptide set forth in either the second or third aspects of the present invention.

According to a fifth aspect of the present invention, there is provided a polynucleotide comprising a base sequence shown in SEQ ID NO: 13, or a base sequence having one or more nucleic acid deletions, additions, insertions or substitutions relative to the base sequence shown in SEQ ID NO: 13. This polynucleotide expresses a polypeptide having a xyloglucan oligosaccharide-degradation activity.

The polynucleotide set forth in the fifth aspect of the present invention may further include an initiation codon.

The polynucleotide set forth in the fifth aspect of the present invention may further include a base sequence corresponding to a signal peptide sequence.

According to a sixth aspect of the present invention, there is provided a polynucleotide comprising a base sequence shown in SEQ ID NO: 11, or a base sequence having one or more nucleic acid deletions, additions, insertions or substitutions relative to the base sequence shown in SEQ ID NO: 11. This polynucleotide expresses a precursor of a polypeptide having a xyloglucan oligosaccharide-degradation activity.

According to a seventh aspect of the present invention, there is provided a polynucleotide which hybridizes under stringent conditions to the polynucleotide set forth in the fifth or sixth aspects of the present invention.

According to an eighth aspect of the present invention, there is provided a polynucleotide having homology to the polynucleotide set forth in any one of the fifth to seventh aspects of the present invention.

According to a ninth aspect of the present invention, there is provided a polynucleotide which is a degenerate of the polynucleotide set forth in any one of the fifth to seventh aspects of the present invention.

According to a tenth aspect of the present invention, there is provided a recombinant vector containing the polynucleotide set forth in any one of the fourth to ninth aspects of the present invention.

According to an eleventh aspect of the present invention, there is provided a transformant containing the recombinant vector set forth in the tenth aspect of the present invention.

According to a twelfth aspect of the present invention, there is provided a method of preparing a polypeptide, wherein the transformant set forth in the eleventh aspect of the present invention is cultured, and then a polypeptide corresponding to a resultingly recombined polynucleotide is collected from the cultured transformant.

In the method set forth in the twelfth aspect of the present invention, the polypeptide may have a xyloglucan oligosaccharide-degradation activity. Further, the xyloglucan oligosaccharide-degradation activity is operative to specifically cleave the second β-glucoside linkage, counted from the reducing end among the β-glucoside linkages constituting the principal chain of xyloglucan oligosaccharide.

The terms “comprising (comprise),” “including (include)” or “having (have),” for example in “polypeptide comprising an amino acid sequence” or “polynucleotide comprising a base sequence” are used herein to encompass not only a polypeptide or polynucleotide consisting of an amino acid or base sequence designated herein, but also any other polypeptide or polynucleotide into which such a designated sequence is incorporated as a part thereof.

The xyloglucan oligosaccharide-degrading enzyme of the present invention has a function of specifically cleaving the second β-glucoside linkage, counted from the reducing end among the β-glucoside linkages constituting the principal chain of xyloglucan oligosaccharide.

This enzyme is preparing from a Geotrichum-species strain M128, which is deposited as Geotrichum sp. strain M128, FERM P-16454 in the International Patent Organism Depositary, National Institute of Advanced Industrial Science and Technology, Japan on Oct. 1, 1997.

Typically, in a process of preparing the enzyme of the present invention from the Geotrichum-species strain M128, the strain is cultured aerobically at 20 to 30° C. for 4 to 10 days by use of a liquid or solid medium which comprises a carbon source consisting of tamarind seed xyloglucan, and a nitrogen source including an inorganic or organic nitrogen source, such as nitrate salt, ammonium salt, peptone or yeast extract, and a small amount of metallic salts. The enzyme of the present invention is an extracellular enzyme excreted from the cells of the strains producing it. Thus, after culturing, a top supernatant obtained from the liquid medium through filtration or centrifugation, or a solution extracted from the solid medium by use of water or suitable inorganic salt, may be used as a crude enzyme solution. This crude enzyme solution contains the enzyme of the present invention and various different types of enzymes having a xyloglucan-degrading activity, and these different types of enzymes should be removed therefrom. These co-existing glycosidase activities can be eliminated through conventional chromatography.

The enzyme (purified product) of the present invention obtained through the above process has the following properties.

(1) Molecular Mass and Isoelectric Point: The purified enzyme of the present invention has a molecular mass of about 96 kDa and an isoelectric point pH of about 6.0.

(2) Action: The purified enzyme of the present invention acts on xyloglucan oligosaccharides having a polymerization degree of 3 or more in the principal chain among xyloglucan-originated oligosaccharides produced by partially degrading xyloglucans of various origins with endo-β-1,4-glucanase, to specifically cleave the second β-glucoside linkage counted from the reducing end among the β-glucoside linkages constituting the principal chain of the oligosaccharide.

If any hydroxyl group other than a 4-hydroxyl group participating in the linkage of the glucose residue at the reducing end of the principal chain is modified, the enzyme of the present invention cannot cleave the target β-glucoside linkage. Further, in the xyloglucan oligosaccharide, if among hydroxyl groups of the xylose side chain a 2-hydroxyl group linked to the third glucose residue counted from the reducing end glucose of the principal chain is modified, the enzyme of the present invention cannot cleave the second β-glucoside linkage counted from the reducing end.

With respect to a cellooligosaccharide having a polymerization degree of 4 or more and no xylose side chain, while the enzyme of the present invention cleaves the second β-glucoside linkage counted from the reducing end to produce cellobiose, the degradation speed or rate is significantly lower than that in a xyloglucan oligosaccharide, and the ratio therebetween is 1:200 or more.

While 1,4-β-D-glucan-cellobiohydrase (EC 3. 2. 1. 91) is known as an enzyme which degrades cellooligosaccharide having a polymerization degree of 4 or more to produce cellobiose, it cannot cleave any β-glucoside linkage constituting the principal chain of xyloglucan oligosaccharide having xylose side chains. As above, no enzyme specifically acting on the second β-glucoside linkage, counted from the reducing end among the β-glucoside linkages constituting the principle chain of xyloglucan oligosaccharide, is known. Thus, the enzyme of the present invention is a novel xyloglucan oligosaccharide-degrading enzyme having a new degradation mechanism.

(3) Acting pH and Optimum pH: When the enzyme of the present invention was applied to xyloglucan hepta-oligosaccharide used as a degradation substrate, at 45° C. for 10 minutes, the observed acting pH and optimum pH of the enzyme were in the range of 3-5 and about 4.0, respectively.

(4) Stable pH Range: The stable pH of the enzyme of the present invention was in the range of 3.5-8.0 on the basis of the remaining activity of the enzyme after it was left at 45° C. for 3 hours in a citrate-phosphate buffer solution.

(5) Action Temperature Range and Optimum Action temperature: When the enzyme of the present invention was applied to xyloglucan hepta-oligosaccharide used as a degradation substrate, at pH 4.0 for 10 minutes, the action of the enzyme was observed at a temperature of about 55° C. or less, and the optimum action temperature was 50° C.

(6) Heat Stability: After the enzyme of the present invention was subjected to a heat treatment under various temperatures for 10 minutes in 50 mM acetate buffer solution (pH 4.0), the activity of the enzyme was fully maintained when the temperature of the heat treatment was 50° C. or less, and about 10% of the activity was lost when the temperature was 55° C. Then, about 85% of the activity was lost when the temperature was 60° C.

(7) Inhibitor: Among various kinds of metal ions, a mercury ion at a concentration of 1 mM or more strongly inhibited the activity of the enzyme of the present invention.

(8) Purification method: The enzyme of the present invention could be purified to have SDS electrophoretic homogeneity by ultrafiltrating a cultivated top supernatant to concentrate and desalinate it, repeatedly subjecting the resulting concentrate to ion-exchange and adsorption chromatography using an anion exchanger PBE 94 column to further concentrate the fractions of the concentrate maintaining the activity, and subjecting the fractions to gel filtration using a TOYOPEAL HW55F column (made by TOSOH Corporation).

(9) Activity Measurement Method: In view of the feature of specifically degrading xyloglucan oligosaccharide, the activity of the enzyme of the present invention was determined by adding an appropriate amount of the enzyme to 0.5 ml (pH 4.0) of 30 mM aqueous solution of xyloglucan hepta-oligosaccharide prepared from tamarind gum while adjusting the total amount the solution to be 1 ml, causing a reaction therebetween at 45° C. for 10 minutes, and measuring the produced reducing-sugar through a Nelson-Somogyi method. The amount of the enzyme capable of reducing one micro-mol of glucose per minute under the above measurement conditions was defined as 1 unit.

The novel xyloglucan oligosaccharide-degrading enzyme is a secretory protein comprising an amino acid sequence shown in SEQ ID NO: 12. In this sequence, the residues 1-23 are a signal sequence which are absent from the mature protein. The amino acid sequence of the mature protein is shown in SEQ ID NO: 14.

Thus, polypeptide suitable as the enzyme of the present invention has at least the amino acid sequence of SEQ ID NO: 14. Any homologous polypeptide comprising an amino acid sequence having one or more amino acid deletions, additions, insertions, or substitutions relative to the amino acid sequence of SEQ ID NO: 14 is also encompassed within the scope of the present invention as long as it has a xyloglucan oligosaccharide-degradation activity.

The polypeptide of the present invention includes a polypeptide precursor comprising the above signal sequence shown in SEQ ID NO: 12, or a homologous polypeptide precursor comprising an amino acid sequence having one or more amino acid deletions, additions, insertions, or substitutions relative to the amino acid sequence shown in SEQ ID NO: 12 and capable of producing a polypeptide having a xyloglucan oligosaccharide-degradation activity through splicing.

The polypeptide of the present invention can also be prepared by means of gene recombination. In this case, the polypeptide may additionally include methionine originated from initiation codon, at the N-terminus of an amino acid sequence shown in SEQ ID NO: 14, or may additionally include an amino acid residue originated from an expression vector, or a histidine tag for purifying a polypeptide to be obtained, at the C-terminus of the amino acid sequence. For example, it was verified that a polypeptide comprising an amino acid sequence shown in SEQ ID NO: 18 had a xyloglucan oligosaccharide-degradation activity.

A polynucleotide encoding the polypeptide of the present invention having a xyloglucan oligosaccharide-degradation activity includes a cloned gene from microorganisms producing the target enzyme, and any gene having homology to the gene. In the homologous gene, the homology may be at least 60% or more, preferably 80% or more, and more preferably 95% or more.

Preferably, the polynucleotide encoding the polypeptide of the present invention having a xyloglucan oligosaccharide-degradation activity is one of the following polynucleotides (DNA or RNA).

(1) A polynucleotide which comprises a base sequence shown in SEQ ID NO: 13, or a homologous base sequence having one or more nucleic acid deletions, additions, insertions, or substitutions, and encoding a polypeptide having a xyloglucan oligosaccharide-degradation activity.

(2) The polynucleotide set forth in the section (1) further including an initiation codon.

(3) The polynucleotide set forth in section (1) which further includes a base sequence corresponding to a signal sequence, or a homologous base sequence having one or more nucleic acid deletions, additions, insertions, or substitutions relative to the above base sequence, and having the capability of expressing a mature protein through splicing. Preferably, this polynucleotide comprises a base sequence shown in SEQ ID NO: 11.

In addition to the above polynucleotides, a polynucleotide which hybridizes under stringent conditions to any one of the polynucleotides set forth in the above sections (1) to (3), a polynucleotide having homology to any one of the above polynucleotides, and a polynucleotide which is a degenerate of any one of the above polynucleotides, are encompassed within the scope of the present invention, as long as polypeptides encoded by these polynucleotides have a xyloglucan oligosaccharide-degradation activity.

The term “stringent conditions” herein means, for example, keeping the polynucleotide at a temperature of 50° C. to 60° C. for 4 hours to one night in a 6×SSC hybridization buffer (1×SSC: 0.15 mol/L, 0.015 mol/L sodium citrate, pH 7.0) containing 0.5% SDS, 5×Denhartd's (Denhartd's: 0.1% bovine serum albumin, 0.1% polyvinyl pyrrolidone, 0.1% Ficoll 400) and 100 μg/ml salmon sperm DNA.

The polynucleotide encoding the polypeptide of the present invention having a xyloglucan oligosaccharide-degradation activity can be obtained by cloning a gene of a microorganism producing the aforementioned xyloglucan oligosaccharide-degrading enzyme, for example, through the following method.

The novel xyloglucan oligosaccharide-degrading enzyme is first isolated from a microorganism producing the novel xyloglucan oligosaccharide-degrading enzyme and purified. Then, information about the partial amino acid sequence of the purified enzyme is obtained.

For example, the partial amino acid sequence may be determined by subjecting the purified enzyme protein directly to an amino acid sequence analyzer (e.g. Protein-sequencer 476A, made by Applied Biosystems) through an Edman degradation method (Journal of Biological Chemistry, 256, 7990-7997 (1981)), or may be effectively determined by subjecting the purified enzyme protein to limited hydrolysis while applying protein hydrolase thereto, isolating and purifying the obtained peptide fragments, and analyzing the amino acid sequence of the purified peptide fragments.

Then, a polynucleotide encoding a novel xyloglucan oligosaccharide-degrading enzyme is cloned in accordance with the obtained information about the partial amino acid sequence. Typically, the cloning can be carried out through a PCR method or a hybridization method.

For the PCR method or hybridization method, the method described in Sambrook and Russell, Molecular Cloning: A Laboratory Manual, Third edition, Cold Spring Harbor Laboratory Press (2001) may be used.

Particularly for the PCR method, the following method may be used. A cDNA of a microorganism producing the new xyloglucan oligosaccharide-degrading enzyme is first prepared as a template. Then, a PCR is performed using a synthesized oligonucleotide primer designed in accordance with the information about the partial amino acid sequence to obtain target polynucleotide fragments.

When the base sequence of the PCR-amplified DNA fragments is determined through a commonly used method such as a dideoxy-chain terminator method, a sequence corresponding to the partial amino acid sequence of the new xyloglucan oligosaccharide-degrading enzyme is found in the determined sequence in addition to the sequence of the synthesized oligonucleotide primer. That is, a part of the polynucleotide encoding the target enzyme can be obtained. Then, a full length polynucleotide encoding the new xyloglucan oligosaccharide-degrading enzyme over its entire length can be cloned through a hybridization method, or a 5′-RACE (Rapid Amplification of cDNA ends) and 3′-RACE method using the obtained polynucleotide fragments as a probe.

In the present invention, the entire base sequence of the polynucleotide encoding the xyloglucan oligosaccharide-degrading enzyme was determined using the Geotrichum sp. strain M128 through a PCR method. This base sequence is shown in SEQ ID NO: 11.

The amino acid sequence encoded by this polynucleotide is shown in SEQ ID NO: 12. The 1st to 23rd amino acid residues counted from the N-terminus of this amino acid sequence indicates a signal sequence. The mature polypeptide has an amino acid sequence starting from the 24th lysine (SEQ ID NO: 14). The base sequence of the polynucleotide encoding this mature polypeptide is shown in SEQ ID NO: 13.

In addition to the base sequences shown in SEQ ID NOs: 11 and 13, additional base sequences corresponding to the amino acid sequences shown in SEQ ID NOs: 12 or 14 exist, due to the redundancy of the genetic code. These additional base sequences each encode the same amino acid sequence as the template sequence. Thus, for example, a degenerate of SEQ ID NO:11 would encode the same amino acid sequence of SEQ ID NO:11. Each of these additional base sequences are encompassed within the scope of the present invention, as well as degenerates of the homologous base sequences of the present invention defined above.

The target polynucleotide can also be obtained through chemical synthesis (Gene, 60(1), 115-127 (1987)) in accordance with the information about the amino acid sequence shown in SEQ ID NOs: 12 or 14 and the base sequence shown in SEQ ID NOs: 11 or 13.

All or a part of the novel xyloglucan oligosaccharide-degrading enzyme polynucleotide comprising the base sequence identified by using the Geotrichum sp. strain M128 may be used as a hybridization probe to select a polynucleotide having high homology to the new xyloglucan oligosaccharide-degrading enzyme polynucleotide comprising the base sequence shown in SEQ ID NO: 11, from a genomic DNA library or cDNA library of microorganisms producing different xyloglucan oligosaccharide-degrading enzymes.

The hybridization may be carried out under the aforementioned stringent conditions. For example, the genomic DNA library or cDNA library obtained from the microorganisms producing the new xyloglucan oligosaccharide is immobilized onto a nylon film, and the prepared nylon film is blocked at 65° C. in a pre-hybridization solution containing a 6×SSC, 0.5% SDS, 5×Denhartd's, and 100 μg/ml salmon sperm DNA. Then, the hybridization solution containing ³²P labeled probes is added to the film, and kept at 65° C. overnight. The nylon film is then rinsed in 6×SSC at room temperature for 10 minutes, in 2×SSC containing 0.1% SDS at room temperature for 10 minutes, and in 0.2×SSC containing 0.1% SDS at 45° C. for 30 minutes, and then subjected to autoradiography. As a result, a polynucleotide which specifically hybridizes to the probe can be obtained. Various types of homologous genes can also be obtained by changing the conditions of the rinse.

A PCR primer can be designed in accordance with the base sequence of the polynucleotide of the present invention. Through a PCR using the obtained primer, a polynucleotide fragment having high homology to the polynucleotide of the present invention can be obtained, and the full length polynucleotide can also be obtained.

In order to determine if the obtained polynucleotide encodes is the target polynucleotide encoding a polypeptide having a xyloglucan oligosaccharide-degradation activity, the presence of xyloglucan oligosaccharide-degradation activity in the obtained polynucleotide may be assumed from the difference or homology in their nucleic acid structures identified by comparing the determined base sequence with the base sequence or amino acid sequence of the new xyloglucan oligosaccharide-degrading enzyme of the present invention. Alternatively, the presence of xyloglucan oligosaccharide-degradation activity in the obtained polynucleotide may be directly measured from a polypeptide prepared from the obtained polynucleotide.

The following method is advantageous to prepare a polypeptide having a xyloglucan oligosaccharide-degradation activity by using the polynucleotide encoding the xyloglucan oligosaccharide-degrading enzyme of the present invention. An expression vector is first preparing comprising the polynucleotide of the present invention, and the obtained recombinant vector containing the polynucleotide is used to transform a host. Then, the obtained transformant is cultured under commonly used conditions to prepare the polypeptide having the new xyloglucan oligosaccharide-degradation activity. The mature polypeptide has no methionine residue at its N-terminus. Thus, the base sequence corresponding to the mature polypeptide may additionally incorporate an initiation codon (atg), and optionally a termination codon (taa/tag/tga) to provide a xyloglucan oligosaccharide-degradation activity in the mature polypeptide.

The host to be used in the above method may be a microorganism, animal cell or plant cell. The microorganism includes: bacteria such as E. coli, Bacillus species, Streptomyces species, and Lactococcus species; yeasts such as Saccharomyces species, Pichia species, and Kluyveromyces species; and filamentous fungi such as Aspergillus species, Penicillium species, and Trichoderma species.

When an eucaryotic cell is used as the host, the mature polypeptide can be prepared through splicing, for example, even if a polynucleotide encoding a precursor polypeptide having the base sequence shown in SEQ ID NO: 11 corresponding to a signal sequence is used as the polynucleotide.

The expression and/or expressed products can be simply determined by use of an antibody to the new xyloglucan oligosaccharide-degrading enzyme. The expression can also be determined by measuring the enzyme activity.

The polypeptide having the new xyloglucan oligosaccharide-degradation activity may be purified from the transformant culture solution through an appropriate combination of centrifugation, UF concentration, salting-out, and various chromatographies such as an ion-exchange resin method, as described above. The polypeptide may also be advantageously purified, for example, by using a polynucleotide including a cay cay cay cay cay cay sequence (SEQ ID NO: 19) to encode the polypeptide to allow a histidine tag to be added to the C-terminus of the polypeptide to be produced, and optionally by using an expression vector having a histidine tag sequence and a termination codon to allow the above polynucleotide to be inserted in the upstream side of the 5′-terminus of the histidine tag.

The primary structure and polynucleotide structure of the xyloglucan oligosaccharide-degrading enzyme have been clarified by the present invention. Thus, the polynucleotide of the present invention can be used to introduce random mutations or site-specific mutations so as to obtain a polynucleotide comprising an amino acid sequence having at least one amino acid deletion, addition, insertion or substitution relative to the amino acid sequence of a natural xyloglucan oligosaccharide-degrading enzyme. This makes it possible to obtain polynucleotides encoding various xyloglucan oligosaccharide-degrading enzymes slightly different from each other in their optimum action temperature, stable action temperature, optimum pH, stable pH, or substrate-specific property while maintaining a desirable xyloglucan oligosaccharide-degradation enzyme activity, and to prepare various polypeptides having these enzyme activities through a genetic engineering process.

The random mutation may be introduced, for example, through a method of chemically treating DNA such as a method of inducing transition mutation or applying sodium bisulfite to substitute a cytosine base with a uracil base (Proceedings of the National Academy of Sciences of the United States of America, 79, 1408-1412 (1982)), a biological method such as a method of inducing base substitutions in the course of synthesizing double-stranded DNA under the presence of [α-S] dNTP (Gene, 64, 313-319 (1988)), or a method using PCR such as a method of adding manganese into a reaction system to perform a PCR so as to provide reduce accuracy in incorporating nucleotides (Analytical Biochemistry, 224, 347-353 (1995)).

The site-specific mutation may be introduced, for example, through a method utilizing amber mutation (gapped duplex method, Nucleic Acids Research, 12(24), 9441-9456 (1984)), a method utilizing a recognition site for a restriction enzyme (Analytical Biochemistry, 200, 81-88 (1992), Gene, 102, 67-70 (1991)), a method utilizing dut (dUTOase) and ung (uracil DNA glycosilase) mutation (Kunkel method, Proceedings of the National Academy of Sciences of the United States of America, 82, 488-492 (1985)), a method utilizing amber mutations using DNA polymerase and DNA ligase (Oligonucleotide-directed Dual Amber (ODA) method, Gene, 152, 271-275 (1995), Japanese Patent Laid-Open Publication No. 7-289262), a method utilizing a host inducing DNA repair system (Japanese Patent Laid-Open Publication No. 8-70874), a method utilizing a protein catalyzing a DNA strand exchange reaction (Japanese Patent Laid-Open Publication No. 8-140685), a PCR method using two kinds of mutagenic primers added with a recognition site for a restriction enzyme (U.S. Pat. No. 5,512,463), a PCR method using two kinds of primers and a double-stranded DNA vector having an inactivated drug-resistance gene (Gene, 103, 73-77 (1991)), and a PCR method utilizing amber mutations (International Patent Application No. WO 98/02535).

The site-specific mutation can be readily induced by using a commercially available kit. The commercially available kit includes Mutan®—G (made by Takara Shuzo Co., Ltd.) utilizing a gapped duplex method, Mutan®—K (made by Takara Shuzo Co., Ltd.) utilizing the a Kunkel method, Mutan®—Express Km (made by Takara Shuzo Co., Ltd.) utilizing an ODA method, and QuikChangeTM Site-Directed Mutagenesis Kit (made by STRATAGENE) using a mutagenic primer and DNA polymerase originated from Pyrococcus furiosus. The commercially available Kit utilizing PCR includes TaKaRa LA PCR in vitro Mutagenesis Kit (made by Takara Shuzo Co., Ltd.) and Mutan®—Super Express Km (made by Takara Shuzo Co., Ltd.).

As described above, the primary structure and polynucleotide structure of the xyloglucan oligosaccharide-degrading enzyme provided by the present invention allows high-purity polypeptides having a xyloglucan oligosaccharide-degradation activity to be prepared at a low cost through a genetic engineering process. The entire contents of the above publications are incorporated herein by reference. While the present invention will be described below in more detail with reference to Examples, the present invention is not limited thereto. The unit % herein means W/V % unless otherwise specified.

### DESCRIPTION OF THE PREFERRED EMBODIMENT

### EXAMPLE 1

20 ml of medium containing 1% of tamarind seed xyloglucan, 0.8% of peptone, 0.05% of magnesium sulfate, 0.2% of monopotassium phosphate, and 0.05% of yeast extract was put in an Erlenmeyer flask having a volume of 200 ml. The culture solution was sterilized in the usual manner, and then the Geotrichum sp. strain M128 (FERM P-16454) was inoculated into the solution. The solution was aerobically cultured at 30° C. for 6 days, and then centrifuged and filtered. The activity of the obtained top supernatant was measured through the aforementioned activity-measurement method. The measured activity per 1 ml culture solution was 0.05 units.

### EXAMPLE 2

4000 ml of culture solution having the same composition as that in EXAMPLE 1 was prepared. The Geotrichum sp. strain M128 was inoculated into the culture solution. The culture solution was cultured in the same way as that in EXAMPLE 1, and the top supernatant of the resulting culture solution was obtained. A total enzyme activity in the culture solution was 216 units. The obtained top supernatant was concentrated and desalinated through ultrafiltration, and then purified through ion exchange/absorption chromatography using an anion exchanger column and gel filtration using a TOYOPEARL HW55F column. The purified enzyme had a SDS-electrophoretic homogeneity and an activity yield of 9.2% in the filtrated culture solution. The activity per 1 mg purified enzyme protein was 11.8 units.

### EXAMPLE 3

0.08 units of the purified enzyme obtained in EXAMPLE 2 was added to 1 ml of reaction solution containing 80 mg of xyloglucan hepta-oligosaccharide, and allowed to react at pH 4.0 and 45° C. for 3 hours. After completion of the reaction, the resulting product was isolated and purified through Bio-Gel P-2 (made by Bio-Rad), and the structure of the purified product was analyzed. The product comprised two kinds of oligosaccharides: an oligosaccharide A having a polymerization degree of 3 and an oligosaccharide B having a polymerization degree of 4. The oligosaccharide A was degraded into equimolar isopremeverose and glucose by an isopremeverose-producing-oligoxyloglucan-degrading enzyme. The oligosaccharide B was degraded by the isopremeverose-producing-oligoxyloglucan-degrading enzyme to form only isopremeverose. This result proved that the enzyme cleaves the second β-glucoside linkage counted from the reducing end of the xyloglucan hepta-oligosaccharide among the β-glucoside linkages constituting the principal chain of the oligosaccharide serving as a substrate, to form branched tetra-saccharide and branched tri-saccharide.

### EXAMPLE 4

0.01 units of the purified enzyme obtained in EXAMPLE 2 was added to 5 mg each of various kinds of xyloglucan oligosaccharides shown in Table 1 in separate vessels, and each combination was allowed to react 18 hours. Then, the resulting oligosaccharides were analyzed through high-speed liquid chromatography. The result proved that the enzyme acts on xyloglucan oligosaccharides comprising a principal-chain having a polymerization degree of 3 or more, and specifically cleaves the second β-glucoside linkage counted from the reducing end among the β-glucoside linkages constituting the principal chain of the oligosaccharide. The result also proved that the enzyme cannot cleave the above second β-glucoside linkage if the glucose residue at the reducing end is modified, and that the side-chain xylose residue linked to the third glucose residue counted from the reducing end glucose of the principal chain has a key role, or the enzyme cannot cleave the above second β-glucoside linkage if the xylose is modified.

TABLE 1

| | | final | | |
|---|---|---|---|---|
| degradation | | degradation | degraded | |
| substrate | structure | ratio (%) | product | |
| penta-oligo- saccharide | 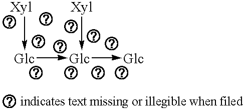 | 100 | 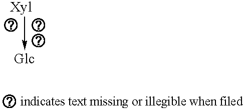 | 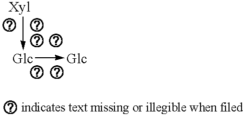 |
| hexa-oligo- saccharide | 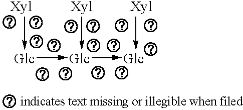 | 0 | NON | |
| hepta-oligo- saccharide | 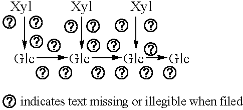 | 100 | 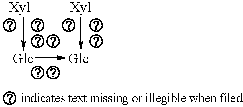 | 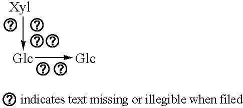 |
| octa-oligo- saccharide | 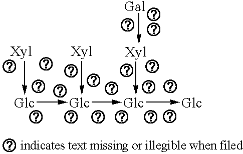 | 100 | 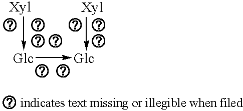 | 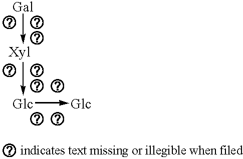 |

| | | | |
|---|---|---|---|
| nona-oligo- saccharide | 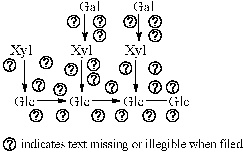 | 0 | NON |

The isopremeverose-producing-oligoxyloglucan-degrading enzyme has been known as one of the enzymes that can directly cleave the 1,4-β-D-glucoside xyloglucan oligosaccharide constituting the principal chain of xyloglucan oligosaccharide having side chains. However, the enzyme degrades xyloglucan oligosaccharide into isopremeverose units in accordance with an exo-type degradation mechanism. The enzyme of the present invention has a completely different substrate-specific property and degradation mechanism from the above conventional enzyme in that it can directly cleave the second β-glucoside linkage counted from the reducing end among the β-glucoside linkages constituting the principal chain of xyloglucan oligosaccharide.

### EXAMPLE 5

Isolation of Polynucleotide Encoding Novel Xyloglucan Oligosaccharide-Degrading Enzyme Originated from Geotrichum sp. Strain M128.

The polynucleotide manipulation technique herein was in accordance with publications (e.g. Sambrook and Russell, Molecular Cloning: A Laboratory Manual, Third edition, Cold Spring Harbor Laboratory Press, 2001) unless otherwise specified.

1. Determination of Partial Amino Acid Sequence

The purified sample of the xyloglucan oligosaccharide-degrading enzyme obtained in EXAMPLE 2 was subjected to Protein Sequencer (made by Applied Biosystems) to determine the 22-residue N-terminus amino acid sequence shown in SEQ ID NO: 1. Then, the purified sample of the xyloglucan oligosaccharide-degrading enzyme obtained in EXAMPLE 2 was degraded with lysyl-end peptidase (made by Wako Pure Chemical Industries, Ltd.). The obtained degradation product was subjected to reversed-phase liquid chromatography to obtain peptide fractions. Three of the separated peptide fractions were subjected to Protein Sequencer to determine the 9 to 12-residue internal amino acid sequences shown in SEQ ID NOs: 2, 3, and 4.

| | |
|---|---|
| SEQ ID NO: 1: | Lys-Glu-His-Tyr-Glu-Phe-Lys-Asn-Val-Ala-Ile-Gly-Gly-Gly-Gly- |
| | Tyr-Ile-Thr-Gly-Ile-Val-Ala |
| SEQ ID NO: 2: | Asp-Leu-Leu-Tyr-Ala-Arg-Thr-Asp-Ile-Gly-Gly-Ala |
| SEQ ID NO: 3: | Ala-Ser-Ala-Pro-Ser-Ala-Val-Phe-Ile-Trp-Gly-Thr |
| SEQ ID NO: 4: | Val-Tyr-Gly-Arg-Val-Tyr-Leu-Gly-Thr |

2. Preparation of Entire RNA

Culturing was conducted in the same way as that in EXAMPLE 1, and RNA was prepared in its entirety in the usual manner using an RNA isolation kit (FastRNA Kit-RED made by BIO 101) as directed by its attached operational instruction. Then, mRNA was purified using an mRNA preparation kit (QuickPrep mRNA Purification Kit made by Amersham Pharmacia Biotech). The obtained mRNA was used to synthesize cDNA with an oligo-dT-primer and reverse transcriptase by use of a cDNA synthesis kit (TimeSaver cDNA Synthesis kit made by Amersham Pharmacia Biotech Inc.).

3. Amplification Through PCR

Based on the N-terminus-region amino acid sequence shown in SEQ ID NO: 1 and the internal amino acid sequence shown in SEQ ID NO: 3, oligonucleotide primers (a sense primer shown in SEQ ID NO: 5 and an antisense primer shown in SEQ ID NO: 6) were synthesized by a DNA synthesizer (made by Applied Biosystems Ltd.). The obtained oligonucleotides were used as PCR primers.

| | |
|---|---|
| Sense Primer: | |
| 5′-GARCAYTAYGARTTYAARAAYGT-3′ | SEQ ID NO: 5 |
| Antisense Primer: | |
| 5′-GTNCCCCADATRAANACNGC-3′ | SEQ ID NO: 6 |

These primers and the cDNA prepared from Geotrichum sp. strain M128 were used in a PCR performed on a GeneAmp PCR system 9700 (made by Applied Biosystems Ltd.).

The PCR solution contained 10 μl of 10×PCR buffer solution (made by Takara Shuzo Co., Ltd.), 8μ of dNTP mixture solution (2.5 mmol/l each, made by Takara Shuzo Co., Ltd.), 5 μl of 100 μmol/L sense primer, 5 μl of 100 μmol/L antisense primer, 71 μl of distilled water, 0.5 μl of cDNA solution (100 μg/ml), and 0.5 μl of EX-Taq DNA polymerase (made by Takara Shuzo Co., Ltd.).

The PCR conditions were as follows:

Stage 1: Denaturation (94° C. for 5 minutes), 1 cycle

Stage 2: Denaturation (94° C. for 1 minute), annealing (45° C. for 1 minute), elongation (72° C. for 2 minutes), 35 cycles

Stage 3: Elongation (72° C. for 10 minutes), 1 cycle

The obtained DNA fragment of about 2 kbp was cloned into the pGEM-T Easy vector (made by Promega), and the base sequence of the cloned DNA was determined. Base sequences encoding the above partial amino acid sequence were found right after the sense primer and right before the antisense primer. A base sequence encoding the internal amino acid sequence shown in SEQ ID NO: 2 was also found.

4. Determination of 5′-Terminus and 3′-Terminus

Based on the above DNA base sequence, two different primers, shown in SEQ ID NOs: 7 and 8, were prepared as antisense primers for 5′-RACE (Rapid Amplification of cDNA ends). cDNA was used as a template to obtain a 5′-terminus DNA fragment through a 5′-RACE method, and the base sequence of the fragment was determined. Similarly, two different primers, shown in SEQ ID NOs: 9 and 10, were prepared as sense primers for 3′-RACE, and a 3′-terminus DNA fragment was obtained through the 3′-RACE method to determine the full length base sequence.

| | |
|---|---|
| SEQ ID NO: 7: | 5′-CGTACAGCAGGTCCTTGGTCTTTGG-3′ |
| SEQ ID NO: 8: | 5′-TAATGTACCCGCCGCCGCCGAT-3′ |
| SEQ ID NO: 9: | 5′-GGCAAGTTCTTCGTCTCGACCGAC-3′ |
| SEQ ID NO: 10: | 5′-CCAAGTCGGACGGCAAGAAGGCTA-3′ |

The determined base sequence of the cDNA encoding the xyloglucan oligosaccharide-degrading enzyme is shown in SEQ ID NO: 11. The obtained cDNA has a total length of 2646 base pairs and includes an open reading frame which extends from an initiation codon (atg) of 0th to 12th residues to a termination codon (taa) of 2556th to 2558th residues, thereby encoding a protein consisting of 812 amino acids, shown in SEQ ID NO: 12. The vicinity of the initiation codon fulfills a requirement of −3−g and +4−g, important for a translation initiation site. The N-terminus amino acid sequence (SEQ ID NO: 1) and the internal amino acid sequences (SEQ ID NOs: 2, 3 and 4) were found in this amino acid sequence.

The sequence (1st to 23rd residues) from the translation initiation site to the site just before the N-terminus amino acid of mature protein (SEQ ID NO: 1) is a signal sequence, which suggests that the enzyme of the present invention is a secretory protein.

The amino acid sequence of the mature protein is shown in SEQ ID NO: 14, and the base sequence of the polynucleotide encoding the mature protein is shown in SEQ ID NO: 13.

The present invention is not limited to polypeptides comprising the above sequences having a xyloglucan oligosaccharide-degradation activity and nucleotides encoding the polypeptides, but any other polypeptides having a longer length and comprising the polypeptide having a xyloglucan oligosaccharide-degradation activity and any nucleotides encoding such polypeptides are encompassed within the scope of the present invention.

### EXAMPLE 6

Construction of Expression Plasmid of New Xyloglucan Oligosaccharide-Degrading Enzyme in E. coli.

Based on the DNA base sequences encoding the N-terminus region amino acid sequence and C-terminus region amino acid sequence of the mature protein, a sense primer shown in SEQ ID NO: 15 and an antisense primer shown in SEQ ID NO: 16 were prepared. During this process, a restriction-enzyme-NdeI recognition sequence (catatg) was added to the 5′ side of the sense primer in the N-terminus region, and a restriction-enzyme-BgMl recognition sequence (agatct) was added to the 5′ side of the antisense primer in C-terminus region. A plurality of bases were added to the 5′ side of the restriction enzyme recognition sequence to provide an increased degradation ratio for the restriction enzyme.

SEQ ID NO: 15:

5′-TCGCTCGCGCATATGAAGGAGCACTACGAGTTCAAGAATG-3′

SEQ ID NO: 16:

5′-TGGAAGATCTCCTTTTTCACATAGCAGTGCGTGCCCTT-3′

These primers, and cDNA prepared from Geotrichum sp. strain M128 for use as a template, were used in a PCR performed using a GeneAmp PCR system 9700 (made by Applied Biosystems Ltd.) under the following conditions.

The PCR solution contained 10 μl of 10×PCR buffer solution (made by Takara Shuzo Co., Ltd.), 8μ of dNTP mixture solution (2.5 mmol/L each, manufactured by Takara Shuzo Co., Ltd.), 5 μl of 10 μmol/L sense primer, 5 μl of 10 μmol/L antisense primer, 71 μl of distilled water, 0.5 μl of cDNA solution (100 μg/ml), and 0.5 μl of EX-Taq DNA polymerase (made by Takara Shuzo Co., Ltd.).

The PCR conditions were as follows:

Stage 1: Denaturation (96° C. for 1 minute), 1 cycle

Stage 2: Denaturation (96° C. for 10 seconds), annealing and elongation (68° C. for 4 minute), 30 cycles

The obtained DNA fragment of about 2.4 kbp was digested by restriction enzymes NdeI and BglII, and cloned into the pET29a(+) vector (manufactured by Novagen, Inc.) digested by restriction enzymes NdeI and BglII. After checking the adequacy of the base sequence, the cloned DNA was introduced into E. coli BL21-CodonPlus (DE3)-RP (made by STRATAGENE). The obtained transformant was shaking-cultured at 37° C. in a LB medium containing 30 μg/ml kanamycin. Isopropyl-β-D-thiogalactopyranoside was added to the medium to induce production. The enzyme accumulated in the bacteria cells in the form of a protein inclusion body.

### EXAMPLE 7

Isolation, Solubilization and Unwinding of Protein Inclusion Body.

The cultivated cells were collected. Then, by using a protein extraction kit (BugBuster manufactured by Novagen), the collected cells were fractured, and the protein inclusion bodies were purified. The purified protein inclusion bodies were dissolved in 8M urea-1 mM dithiothreitol-50 mM Tris HCl-1 mM ethylenediamine tetraacetic acid (pH 8.0) to adjust a protein concentration to about 1 mg/ml.

The solubilized top supernatant was dialyzed with 25 mM imidazole-HCl (pH 7.4) to remove urea and dithiothreitol so as to unwind polypeptide therein.

The amino acid sequence of the obtained polypeptide is shown in SEQ ID NO: 18, and the base sequence corresponding to this amino acid sequence is shown in SEQ ID NO: 17. The polypeptide comprises the amino acid of the mature polypeptide shown in SEQ ID NO: 14 having a C-terminus which additionally includes a histidine tag comprising 30 amino acid residues originated from the expression vector pET29a (+) and 6 histidine residues. The xyloglucan oligosaccharide-degradation activity of the polypeptide was examined by using the various xyloglucan oligosaccharides shown in Table 1 as a substrate. The results were completely the same as that of the enzyme obtained from the Geotrichum sp. strain M128. That is, the recombinant enzyme had an enzyme activity of specifically cleaving the second β-glucoside linkage counted from the reducing end among the β-glucoside linkages constituting the principal chain of the xyloglucan oligosaccharide.

From the above results, it was verified that the polynucleotide of the new xyloglucan oligosaccharide-degrading enzyme of the present invention can be used to prepare a recombinant enzyme using E. coli.

As mentioned above, the present invention provides a new enzyme which cleaves the second β-glucoside linkage, counted from the reducing end among the β-glucoside linkages constituting the principal chain of xyloglucan oligosaccharide. Further, the present invention discloses the amino acid sequence and the polynucleotide structure of the new enzyme. Thus, the present invention opens the way for achieving a new analysis technique of analyzing a structure and function of xyloglucan which must play an important role in elongation and morphological differentiation of plant cells.

What is claimed is:

1. A purified enzyme which specifically cleaves the second β-glucoside linkage counted from the reducing end among the β-glucoside linkages constituting the principal chain of xyloglucan oligosaccharide.

2. A purified polypeptide comprising the amino acid sequence shown in SEQ ID NO: 14, or an amino acid sequence having one or more amino acid residue deletions, additions, insertions or substitutions relative to the amino acid sequence shown in SEQ ID NO: 14, said polypeptide having a xyloglucan oligosaccharide-degradation activity.

3. The purified polypeptide as defined in claim 2, which further includes methionine at the N-terminus thereof.

4. The purified polypeptide as defined in claim 2, which further includes a signal sequence.

5. A purified polypeptide comprising the amino acid sequence shown in SEQ ID NO: 12, or an amino acid sequence having one or more amino acid deletions, additions, insertions or substitutions relative to the amino acid sequence shown in SEQ ID NO: 12, said polypeptide serving as a precursor of a polypeptide having a xyloglucan oligosaccharide-degradation activity.

6. The purified polypeptide as defined in claim 3, wherein said amino acid sequence is the amino acid sequence shown in SEQ ID NO: 18.

7. An isolated polynucleotide encoding the polypeptide as defined in any one of claims 2 to 5.

8. An isolated polynucleotide comprising the base sequence shown in SEQ ID NO: 13, or a base sequence having one or more nucleic acid deletions, additions, insertions or substitutions relative to the base sequence shown in SEQ ID NO: 13, said polynucleotide encoding a polypeptide having a xyloglucan oligosaccharide-degradation activity.

9. The isolated polynucleotide as defined in claim 8, which further includes an initiation codon.

10. The isolated polynucleotide as defined in claim 8, which further include a base sequence corresponding to a signal peptide sequence.

11. An isolated polynucleotide comprising the base sequence shown in SEQ ID NO: 11, or a base sequence having one or more nucleic acid deletions, additions, insertions or substitutions relative to the base sequence shown in SEQ ID NO: 11, said polynucleotide encoding a precursor of a polypeptide having a xyloglucan oligosaccharide-degradation activity.

12. An isolated polynucleotide which hybridizes to a polynucleotide as defined in any one of claims 8 to 11, under stringent conditions.

13. An isolated polynucleotide having homology to a polynucleotide as defined in any one of claims 8 to 11.

14. An isolated polynucleotide which is a degenerate of a polynucleotide as defined in any one of claims 8 to 11.

15. A recombinant vector comprising a polynucleotide as defined in any one of claims 8 to 11.

16. A transformant comprising the recombinant vector as defined in claim 15.

17. A method of preparing a polypeptide, said method comprising culturing the transformant as defined in claim 16 under conditions such that said transformant produces the polypeptide encoded by the polynucleotide, and collecting the polypeptide so expressed.

18. The method as defined in claim 17, wherein said polypeptide has a xyloglucan oligosaccharide-degradation activity.

19. The method as defined in claim 18, said xyloglucan oligosaccharide-degradation activity is operative to specifically cleave the second β-glucoside linkage counted from the reducing end among the β-glucoside linkages constituting the principal chain of xyloglucan oligosaccharide.

20. A purified polypeptide consisting of amino acid residues 1 to 789 of SEQ ID NO: 14.

21. A purified polypeptide consisting of amino acid residues 1 to 812 of SEQ ID NO: 12.

22. An isolated polynucleotide consisting of nucleic acid residues 1 to 2367 of SEQ ID NO: 13.

23. An isolated polynucleotide consisting of nucleic acid residues 1 to 2646 of SEQ ID NO: 11.
